(19) United States
(12) Patent Application Publication
(10) Pub. No.: US 2005/0260282 A1
(43) Pub. Date: Nov. 24, 2005

(54) Novel hypoglycemic agent, diabetes preventive agent, deep sea water concentrate, and method for producing deep sea water concentrate

(75) Inventors: Akira Tuji, Kanazawa-shi (JP); Yoshinobu Kozuka, Kanazawa-shi (JP); Yoshimasa Shimbo, Kanazawa-shi (JP)

Correspondence Address: OLIFF & BERRIDGE, PLC, P.O. BOX 19928, ALEXANDRIA, VA, 22320, US

(73) Assignee: Yoshinobu KOZUKA, Kanazawa-shi (JP)

(21) Appl. No.: 10/939,510

(22) Filed: Sep. 14, 2004

(30) Foreign Application Priority Data
May 21, 2004 (JP) 2004-151828

(51) Int. Cl. A61K 33/14

(52) U.S. Cl. 424/680

(57) ABSTRACT

A hypoglycemic agent includes a concentrate obtained by concentrating deep sea water by heating under reduced pressure. A diabetes preventive agent includes a concentrate obtained by concentrating deep sea water by heating under reduced pressure.

Japanese Patent Application No. 2004-151828, filed on May 21, 2004, is hereby incorporated by reference in its entirety.

### BACKGROUND OF THE INVENTION

The present invention relates to a hypoglycemic agent and a diabetes preventive agent using seawater collected from the deep sea (hereinafter called “deep sea water”), a deep sea water concentrate, and a method for producing a deep sea water concentrate.

Mineral components and sea bacteria differing from those contained in normal seawater are found in seawater in the deep sea at a depth of 200 to 300 meters or more. Therefore, the deep sea water is being used not only for drinking water, but also for various types of food processing.

In this case, it is necessary to concentrate or desalt the deep sea water.

As a method for concentrating the deep sea water, an ion-exchange method (Japanese Patent Application Laid-open No. 2001-211864) and an electrolysis method (Japanese Patent Application Laid-open No. 2000-236464) have been proposed. However, these methods do not allow the deep sea water to fully exhibit the original efficacy.

Japanese Patent Application Laid-open No. 2003-63969 has disclosed a technology relating to functional goods using a mineral concentrate separated from the deep sea water, for example.

However, the concentration method described in Japanese Patent Application Laid-open No. 2003-63969 is based on electrodialysis using an ion-exchange membrane, and monovalent cations and anions are removed from the mineral concentrate used for the invention.

The mineral concentration method using electrodialysis is effective for concentrating specific minerals. However, since the physiological adjustment function components of the deep sea water are removed, the deep sea water does not fully exhibit the efficacy.

From ancient times, salt has been obtained by evaporating and condensing the water contained in seawater and further heating the concentrated seawater.

It is a common technology to use steam for heating liquid.

In the case of steam heating, if the temperature of the steam for heating is not higher to a certain extent than the boiling temperature of the liquid to be heated, a prompt heat transfer cannot be achieved, whereby efficient processing cannot be performed.

When evaporating the water contained in seawater by steam heating, since the temperature of the steam for heating must be higher than the boiling point of water, pressurized steam is used.

Therefore, since pressure vessels are used as a heating vessel, a boiler used for steam generation, and the like, strength or airtightness must be taken into consideration. Moreover, since the seawater is boiled at a high temperature, the physiological effect of the deep sea water may be lost.

### BRIEF SUMMARY OF THE INVENTION

The present invention may provide a novel hypoglycemic agent and a diabetes preventive agent containing a concentrate obtained by a concentration method which allows the deep sea water to fully exhibit the physiological effect.

The present invention may further provide a deep sea water concentrate and a method for producing the same by which the deep sea water fully exhibits the physiological effect.

A hypoglycemic agent according to one aspect of the present invention includes a concentrate obtained by concentrating deep sea water by heating under reduced pressure.

A diabetes preventive agent according to another aspect of the present invention includes a concentrate obtained by concentrating deep sea water by heating under reduced pressure.

A deep sea water concentrate according to a further aspect of the present invention is obtained by concentrating deep sea water by heating under reduced pressure and has a sodium chloride content of 15 mass % or more. With this deep sea water concentrate, the decompression condition may be 0.5 atmospheres or less.

A concentration method and an adjustment method for the deep sea water according to the present invention are described below.

The deep sea water was collected from a depth of about 300 meters in the Sea of Japan offshore from Uchiura-machi, Ishikawa, Japan.

The collected deep sea water was placed in a concentrator provided with a rotational drum type decompression chamber, and was concentrated by steam heating from the periphery under reduced pressure.

The decompression condition affects the steam heating temperature, concentration, and concentration time. The deep sea water easily boils at a low temperature by increasing the degree of vacuum in the decompression chamber. However, if the deep sea water boils too violently, the components may be mixed into the distilled water.

The degree of vacuum may be comparatively decreased in order to increase the concentration rate.

Therefore, the decompression condition may be 0.5 atmospheres or less. The deep sea water may be slowly concentrated through boiling at a low temperature while increasing the degree of vacuum.

The steam was used as the heat source because the heating temperature can be prevented from being excessively increased and the decompression chamber can be efficiently and uniformly heated from the outside over the circumference.

Heated steam is obtained at normal pressure by high-frequency heating saturated steam obtained from a boiler or the like by causing the saturated steam to pass through a pipe provided with a wound high-frequency coil. It is efficient to use such heated steam as the heat source of the decompression chamber.

The above-described example of using heated steam as the heat source when concentrating the deep sea water by heating under reduced pressure is an example which enables efficient heating.

Therefore, the heat source is not limited, and electricity, gas, or the like may be used.

The deep sea water concentrated to such a concentration that the sodium chloride content in the deep sea water was about 15 to 18 mass % was used as an undiluted solution.

In this specification, deep sea water with a sodium chloride content of 15 mass % or more is called high concentration deep sea water.

In the present invention, since the deep sea water is concentrated by heating under reduced pressure, the deep sea water can be concentrated at a low temperature while sufficiently maintaining the physiological effect. Therefore, an excellent hypoglycemic effect can be obtained.

In the case where a human or an animal orally takes the deep sea water of the present invention, it is preferable to take a diluted solution obtained by diluting the deep sea water concentrate (undiluted solution) 100 to 300 times, and still more preferably 200 to 300 times from the viewpoint of ease of intake.

### BRIEF DESCRIPTION OF THE SEVERAL VIEWS OF THE DRAWING

FIG. 1 shows a measurement result for the blood glucose level.

FIG. 2 shows a measurement result for the weight.

FIG. 3 shows a measurement result for food consumption (per week).

FIG. 4 shows a measurement result for water intake (per week).

FIG. 5 shows a component analysis result for a deep sea water concentrate (undiluted solution) and a 200-fold diluted solution (administration solution).

### DETAILED DESCRIPTION OF THE EMBODIMENT

The deep sea water collected from a depth of about 300 meters offshore from Uchiura-machi, Ishikawa, Japan was placed in a concentrator provided with a rotational drum type decompression chamber, and was slowly concentrated by steam heating from the periphery under reduced pressure of 0.5 to 0.1 atmospheres or less while rotating the decompression chamber until the sodium chloride content was about 15 to 18 mass %. Salt starts to precipitate when the deep sea water is concentrated to a sodium chloride content of about 18 mass % or more.

A solution obtained by diluting the concentrated undiluted solution about 200 times was used for an investigation of the physiological effect on mice.

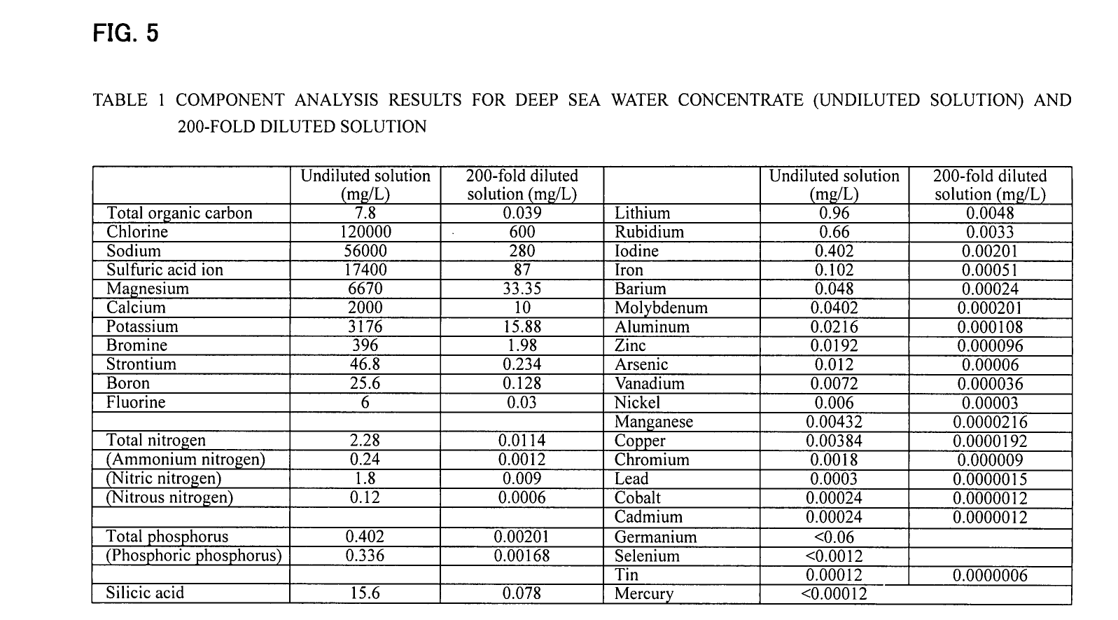

The compositions of the concentrated undiluted solution and the 200-fold diluted solution (mouse administration solution) are shown in FIG. 5 (Table 1).

The pH of the undiluted solution was 8.3.

As shown in FIG. 5, the undiluted solution contains a larger amount of various minerals in comparison with normal seawater.

It is readily expected that other components which are not shown in the component table are contained in the deep sea water.

The investigation results for the pharmacological effect of a hypoglycemic agent according to the present invention using the deep sea water on mice are described below.

KK-A^{Y}/TaJcI mice (male, five weeks old) were individually kept in cages.

The first week was used for preliminary breeding for allowing the mice to adapt themselves to the facility, in which the mice were administered food (CE-2) (manufactured by CLEA Japan, Inc.) and deep sea water obtained by diluting the above concentrated undiluted solution 200 times (hereinafter called “deep sea water”), but the blood glucose level was not measured. The mice as a control were administered food (CE-2) (manufactured by CLEA Japan, Inc.) and normal breeding water.

The measurement of the blood glucose level was started when the mice were six weeks old.

Blood was collected from the caudal vein (about 2 to 5 μL) at a predetermined time each week, and the blood glucose level was measured (“Glutest Ace R” manufactured by Kyoto Daiichi Kagaku, Co., Ltd.).

After measuring the blood glucose level, the weight, food consumption, and water intake of each mouse were measured.

In order to accurately measure the water intake, the amount of water in the water bottle was adjusted to 150 mL, and the water intake was calculated from the residual amount of water after one week.

The amount of food administered each time and the residual amount after one week were recorded, and the food consumption was calculated by subtraction.

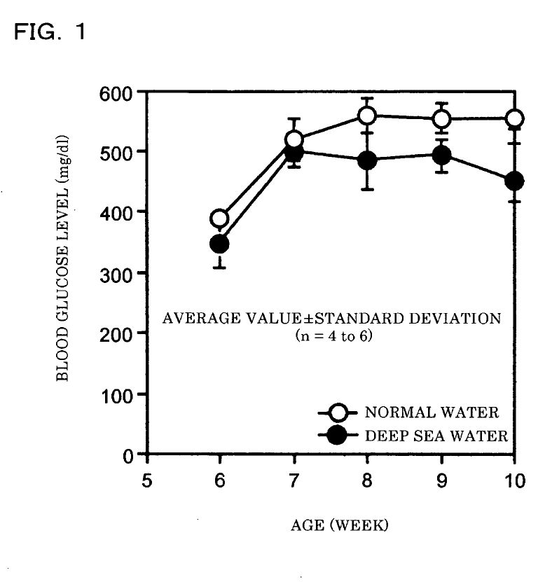

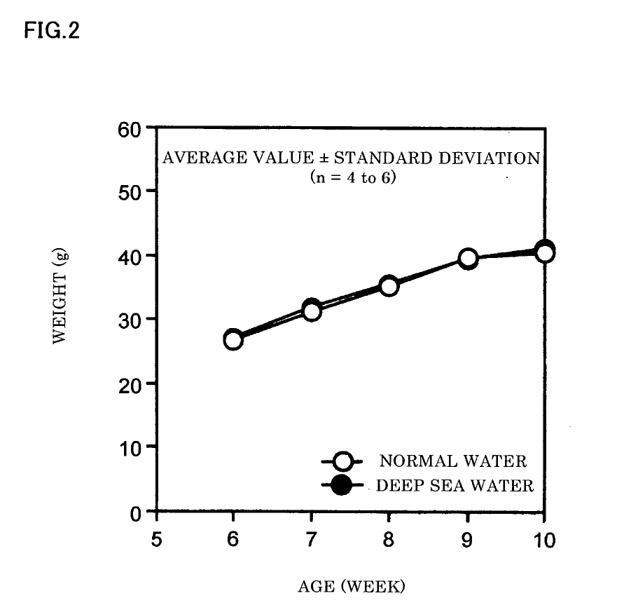

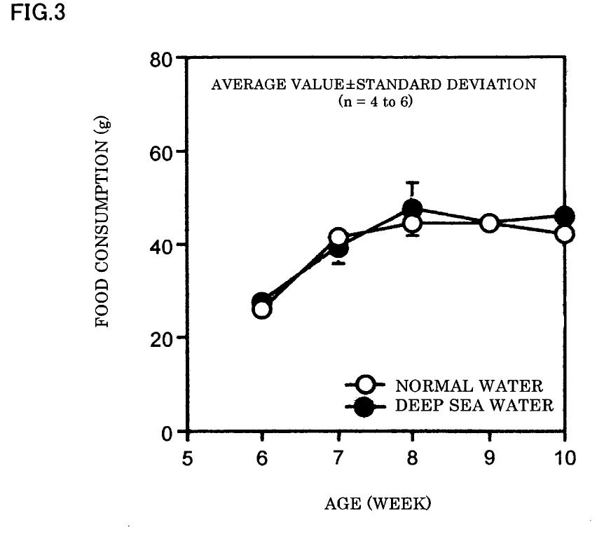

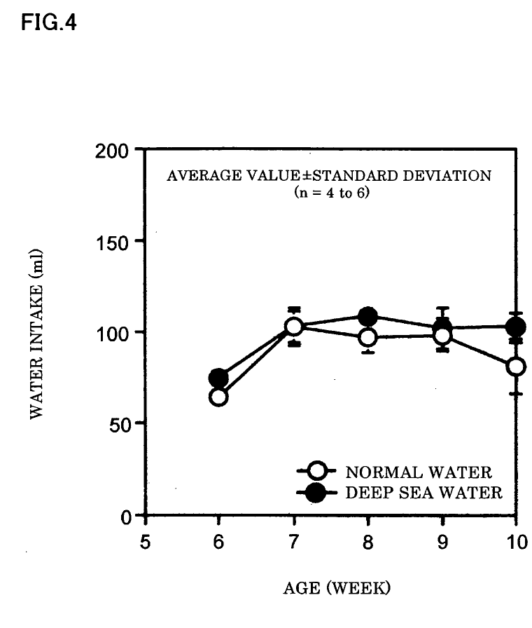

FIG. 1 (Graph 1) shows changes in blood glucose level, FIG. 2 (Graph 2) shows changes in weight, FIG. 3 (Graph 3) shows food consumption, and FIG. 4 (Graph 4) shows water intake.

As a result, no differences were observed for the food consumption, water intake, and weight. However, it was found that the blood glucose level was decreased according to the graph shown in FIG. 1.

The mice used for the investigation had a high blood glucose level, and the blood glucose level was increased to 550 mg/dl by normal breeding using normal water. However, in the mice administered the hypoglycemic agent according to the present invention using the deep sea water, not only was an increase in the blood glucose level prevented, but also the blood glucose level was gradually decreased.

Therefore, the difference is considerable.

The data shown in the Graphs 1 to 4 indicate the average value and the standard deviation of the six mice administered the deep sea water, and the average value and the standard deviation of the four mice administered normal water as a control.

The changes in weight and the like were not observed among individuals to such an extent that the standard deviation could not be shown in the Graph 2.

These results suggest that the deep sea water concentrated under reduced pressure exhibits a hypoglycemic effect. Therefore, it is expected that the deep sea water can be applied to a superior hypoglycemic agent and diabetes preventive agent.

1. A hypoglycemic agent comprising a concentrate obtained by concentrating deep sea water by heating under reduced pressure.

2. A diabetes preventive agent comprising a concentrate obtained by concentrating deep sea water by heating under reduced pressure.

3. A deep sea water concentrate obtained by concentrating deep sea water by heating under reduced pressure and having a sodium chloride content of 15 mass % or more.

4. The deep sea water concentrate according to claim 3, wherein the decompression condition is 0.5 atmospheres or less.
